(19) United States
(12) Patent Application Publication
(10) Pub. No.: US 2003/0153056 A1
(43) Pub. Date: Aug. 14, 2003

(54) Glycosyltransferase proteins

(76) Inventors: Stephen Cohen, Heidelberg (DE); Katja Bruckner, Boston, MA (US); Henrik Clausen, Holte (DK); Birgit Keck, Neuss (DE)

Correspondence Address: WENDEROTH, LIND & PONACK, L.L.P., 2033 K STREET N. W., SUITE 800, WASHINGTON, DC, 20006-1021, US

(21) Appl. No.: 10/276,610

(22) PCT Filed: May 21, 2001
(86) PCT No.: PCT/IB01/01033

(30) Foreign Application Priority Data
May 19, 2000 (GB) 0012216.8

(51) Int. Cl. C12P 21/06; C12P 19/18; C12N 9/99; C12N 9/10

(52) U.S. Cl. 435/97; 435/184; 435/193; 435/68.1

(57) ABSTRACT

The invention is based on the discovery that various proteins, such as Fringe, Brainiac and homologues and orthologues thereof, possess glycosyltransferase activity. Fringe and Brainiac have been found to possess glycosyltransferase activity in transfering sugar residues onto certain proteins, so affecting the binding of effector molecules to these proteins. This discovery allows the design of drug molecules that specifically target this interaction, and has implications for the treatment of various diseases.

The present invention is based on the discovery that various proteins act as glycosyltransferase enzymes that modify certain glycoproteins and glycolipids. In particular, the proteins Fringe and Brainiac have been found to possess glycosyltransferase activity in transferring sugar residues onto certain proteins of biological interest, so affecting the binding of effector molecules to these proteins. Brainiac also transfers sugars onto glycolipids. This discovery allows the design of drug molecules that specifically target these interactions, and has implications for the treatment of various diseases.

In Drosophila, the Fringe protein is known to modify the receptor protein Notch. The Notch family of transmembrane proteins forms a highly conserved group of molecules that serve as receptors for cell to cell communication in both vertebrate and invertebrate organisms. The Notch protein itself is a large (>300 kDa) cell-surface receptor that mediates developmental cell-fate decisions. The protein is known to be essential in a wide variety of developmental cascades including neurogenesis, mesoderm formation, somite formation angiogenesis, germ line and ovarian follicle development, larval Malphigial tubule formation, sensory structure differentiation, eye development, limb formation and lymphoid development.

Homologous components of the Notch signalling pathway are present in organisms ranging from Caenorhabditis elegans to humans. However, much of the research performed to date on this protein family has been in Drosophila, due to the ease of manipulation and study of this organism. Drosophila contains just one Notch homologue, but at least four Notch homologues are present in humans. These proteins are designated Notch1-4.

Notch signalling is induced upon binding to cell-surface ligands such as Delta and Serrate on adjacent cells. Notch contains 36 tandem epidermal growth factor (EGF) modules comprising the majority of its extracellular domain. These EGF modules have been hypothesised to be involved in ligand binding (Fleming et al., (1997). Defects in Notch signalling caused by mutations in these EGF modules have been implicated in various human disease states, including T cell leukaemia, breast cancer, stroke, dementia, cerebral autosomal dominant arteriopathy and leukoencephalopathy.

Ligands that are capable of activating Notch-family receptors are broadly expressed in animal development, yet their activity is tightly regulated to allow formation of tissue boundaries. Members of the Fringe gene family have been implicated in limiting Notch activation during boundary formation, but the mechanism of Fringe function has not been determined. In the developing Drosophila wing, asymmetric activation of Notch by the dorsally expressed ligand Serrate and the ventrally-expressed ligand Delta is required to induce Wingless and Vestigial expression and to establish a signalling centre at the dorsal-ventral boundary. Fringe has been shown to be expressed in dorsal cells and contributes to making these cells more sensitive to Delta and less sensitive to Serrate.

Brainiac codes for a putative secreted protein that has been implicated as a modulator of the activities of both the EGF receptor and of Notch, implicating this protein in a number of developmental events. Various studies have suggested that Brainiac is specifically required for epithelial development (Goode et al., (1996). In addition to being required zygotically for segregation of neuroblasts from epidermoblasts, it is essential for a series of critical steps during oogenesis.

However, the mechanism by which Fringe and Brainiac modulate the activity of target proteins is presently unknown. One means by which these proteins have been suggested to change the cells' sensitivity to Notch ligands is by directly modulating the ligand-receptor interaction, perhaps by acting as co-receptors (Ju et al. 2000). Alternatively, the proteins have been proposed to act directly to influence cellular signalling responses to a given level of ligand binding.

Due to the important role of the Notch family of proteins in cellular processes and their implication as disease-causing agents when functioning aberrantly or when incorrectly expressed, there is a great need for methods for inhibiting the interaction of Notch with its effector ligands.

The aim of the present invention is to explain the factors that govern the interaction of Notch with its ligands, thus paving the way for the development of agents that are effective in modulating this interaction.

### SUMMARY OF THE INVENTION

According to the invention, there is provided the use of a Fringe protein or a Brainiac protein, or a fragment, or functional equivalent of a Fringe protein or a Brainiac protein, as a glycosyltransferase.

The inventors have discovered that Fringe acts in the Golgi as a glycosyltransferase enzyme that modifies the ability of Notch to bind its ligand Delta. Fringe is shown herein to catalyze the addition of N-acetylglucosamine to EGF modules of Notch, suggesting a role in biosynthetic pathways of Fucose O-glycosylation associated with EGF repeats. Brainiac has also been shown to possess a glycosyltransferase activity. As a result of these discoveries, it is postulated that cell-type specific modification of glycosylation may provide a general mechanism to regulate receptor ligand interaction in vivo.

By “Fringe” protein is meant any protein in the Fringe protein family. The importance of these proteins is illustrated in part by their ubiquitous nature, occurring as they do in organisms as diverse as the invertebrate C. elegans and in humans. In Drosophila, the following proteins are presently known to be members of the Fringe protein family (the proteins are identified by their GenBank accession codes): gb|AAF51197.1| (AE003581) CG2975 gene product; gb|AAF48479.1| (AEB003499) CG9220 gene product; gb|AAF52723.1| (AE003623) CG9520 gene product; gb|AAF51199.1| (AE003581) CG3119 gene product; gb|AAF59121.1| (AE003838) CG8708 gene product; gb|AAF47429.1| (AE003469) CG13904 gene product; gb|AAF48917.1| (AE003511) CG7440 gene product; gb|AAF51193.1|(AE003581) CG2983 gene product.

By “Brainiac” protein is meant any protein in the Brainiac protein family. Like Fringe, Brainiac proteins are also thought to exist in a wide variety of different organisms. In Drosophila, the following proteins are presently known to be members of the Brainiac protein family (the proteins are identified by their GenBank accession codes): gb|AAF48225.1| (AE003491) CG4351 gene product; gb|AAF52606.1| (AE003620) CG8668 gene product; gb|AAF47918.1| (AE003481) CG11357 gene product; gb|AAF58600.1| (AE003824) CG8976 gene product; gb|AAF59065.1| (AE003836) CG8734 gene product; gb|AAF59121.1| (AE003838) CG8708 gene product; gb|AAF47429.1| (AE003469) CG13904 gene product; gb|AAF48225.1| (AE003491) CG435 1gene product.

According to the invention, included as Fringe and Brainiac proteins are proteins that are in the same protein family as these proteins. By protein family is meant that the proteins exhibit common features of sequence or structure that indicate that the proteins share a common biological function and/or activity, and have diverged from a common ancestor.

PSIBAST searches with Brainiac and Fringe as queries have been performed until convergence to scan the nonredundant database at the National Collection for Biological Information (NCBI). After iteration, Impala profiles (Schaffer et al., 1999) were built and scanned against Wormpep 20 (http://www.sanger.ac.uk/Projects/C_elegans/wormpep/) and the complete protein complement of the Drosophila genome. A multiple alignment of all protein-family members was built using Clustal (Jeanmougin et al., 1998) and manually refined to maximize residue conservation and imply a consistent secondary structure across the entire family of proteins. The results are shown in FIG. 1; all of the proteins identified in this Figure are included as members of the Fringe and Brainiac protein families. Of course, proteins that are subsequently identified that possess structural or sequence characteristics in common with these protein families are also intended to be included within the term “protein family” as this term is identified herein.

Also shown, as FIG. 2, is a diagrammatic representation of the Fringe and Brainiac families in the form of a phylogenetic tree (Jeanmougin et al., 1998). The genes are identified by both species and identifier. The numbers on the chart are numerical values that provide a measure of the reliability of the placement of the entry to the right of the number. Higher values indicate higher reliability. Any value over 70 is considered highly reliable; accordingly, proteins with values of higher than 70 are considered particularly preferred proteins for use in the present invention. This tree thus serves to identify accurately the currently predictable (or known) family members and can also be taken as the method for identifying potential new family members.

Fragments of Fringe and/or Brainiac proteins are also included within the definition of the present invention, provided that these fragments retain functional biological activity as glycosyltransferase enzymes. Functional glycosyltransferase activity may be assessed by way of a biological assay, either in vitro or in vivo and is defined herein as the ability of a protein fragment to transfer a glycosyl moiety in the form of a UDP-linked donor sugar onto an acceptor sugar or oligosaccharide, whether free or attached to a lipid, carbohydrate or protein.

Fringe is thought to function primarily as an enzyme adding an additional sugar to already fucosylated protein to produce N-acetylglucosamine-fucose-o-Ser/Thr. Fucose is commonly found as an unsubstituted terminal sugar residue in N- and O-linked oligosaccharide chains of glycoproteins and in glycosphingolipids of eukaryotic cells. In contrast, addition of O-linked Fucose directly to proteins is a rare type of glycosylation that is found in association with the cysteine-rich consensus sequence C-x-x-G-G-S/T-C (see Harris and Spellman, 1993). Elongation of the O-linked Fucose occurs only in a subset of proteins that are modified by this type of glycosylation. O-linked Fucose may be extended by addition of β1-3 N-acetyl glucosamine followed by addition of galactose and sialic acid residues. This carbohydrate modification is found on the EGF-like modules of several secreted proteins, including urokinase, tissue-type plasminogen activator, the clotting factors VII and XII that are involved in blood coagulation and fibrinolysis, and recently described in mammalian Notch (Moloney et al. 2000). An as yet unidentified enzyme carries out the first reaction of linking a fucose residue to a protein. Interestingly, a protein termed peptide O-fucosyltransferase, that is responsible for the addition of O-linked fucose to protein, has been characterized in Chinese hamster ovary cells (Wang and Spellman, 1998).

O-Linked fucose is now known to exist as both a monosaccharide and an elongated species. For example, Human clotting factor IX has been reported to contain O-linked fucose elongated into a tetrasaccharide with the structure Sia-α2,6-Gal-β1,4-GlcNAc-β1,3-Fuc-α1-O-Ser (Harris et al., 1993; Nishimura et al., 1992) and O-linked fucose has been shown to be elongated into the disaccharide Glc-β1,3-Fuc-1-O-Ser/Thr (Moloney et al., 1997). All other known O-linked fucose proteins reported to date bear a single monosaccharide species. It is therefore thought that Fringe may elongate fucose through the addition of a β-linked glucose to form the disaccharide or, alternatively, through the addition of a β-linked N-acetylglucosamine, which subsequently results in the formation of the tetrasaccharide. O-Linked glucose in all cases has been shown to be elongated by either one or two xyloses to yield a di- or trisaccharide, respectively (Nishimura et al., 1989).

Brainiac has been found to transfer to free fucose with activity and acceptor substrate titration curves that are identical to Fringe. However, Brainiac appears to have different functions than Fringe in general, and it is demonstrated herein that Brainiac, in contrast to Fringe, has GlcNAc-transferase activity with mannose as acceptor. Without wishing to bound by this theory, the inventors hypothesise that in vivo, Brainiac may create GlcNAcβ1-3Manβ1-4Glcβ1-O-Ser in the EGF repeats of Notch, near the O-linked Fuc sites (the consensus O-Glc and O-Fuc are conserved in the left side of the repeat sequence around loop 1 and 2: CASFPCQNGGTC consensus Glc: CxSxPC; Consensus Fuc: CxxGGTC). Thus, it is hypothesised that in vivo, while Fringe acts at the Fucose sites, Brainiac probably modifies the Glucose sites and hence can be predicted to create related but yet distinct signals for Notch activity. Mammalian Brainiac is also inferred to catalyze linkage of GlcNAc to alpha-Mannose on O-linked mannose found in brain glycoproteins of mammals as Brainiac shows activity with Manα1-MeUmb. Furthermore, as βmannose structures were identified as preferred substrates, in vivo Brainiac may create GlcNAcβ1-3Manβ1-4Glcβ1-Cer containing glycosphingolipid species. The effect that Brainiac may exert in glycosphingolipid biosynthesis can directly or indirectly modulate Notch activity. This is inferred from the well-studied phenomena of ganglioside mediated modulation of EGF receptor activity and phosphorylation as well as the role that glycolipids may exert on proteins in lipid microdomains (see Hakomori and Igarashi (1995), J. Biochem. (Tokyo); 118:1091-103.; Meuillet et al., 2000, Exp. Cell Res.;256: 74-82.; and Hakomori (2000) Glycoconj J.;17:143-51).

The only known enzymes responsible for the addition and elongation of O-linked glucose are the peptide O-glucosyltransferase (Shao et al., 1998), the O-glucose-1,3-xylosyltransferase (Omichi et al., 1997) and the xylose-1,3-xylosyltransferase (Minamida et al., 1996). A protein termed peptide O-fucosyltransferase, that is responsible for the addition of O-linked fucose to protein, has been characterized in Chinese hamster ovary cells (Wang and Spellman, 1998). In addition, the enzyme responsible for the synthesis of the O-linked fucose disaccharide has been identified (O-fucose 1,3-glucosyltransferase; Moloney and Haltiwanger, 1999). These enzymes are found in a variety of tissues and species, suggesting that these carbohydrate modifications are widespread in biology.

Targets for the glycosyltransferase activity of Fringe and Brainiac include glycosylation sites in EGF-like modules that occur in various protein sequences. The EGF-like module is a sequence of thirty to forty amino-acid residues which has significant homology to epidermal growth factor (EGF). It is found in single or multiple copies in a number of other proteins, generally in the extracellular domain. The distinctive features of the EGF module are six conserved cysteine residues, that form three intramolecular disulphide bridges, so giving this module a distinct three-dimensional fold. In regions other than the conserved cysteine residues, the sequences of different EGF modules are highly divergent.

Any protein that contains one or more EGF-like modules and that contains a consensus site for O-linked fucosylation may provide a suitable target for Fringe or for Brainiac. Examples include the EGF receptor, urokinase (Kentzer et al., 1990), tissue-type plasminogen activator (Harris et al., 1991), Factor VII (Bjoern et al., 1991); Factor IX (Nishimura et al., 1992), Factor XII (Harris et al., 1992) and foetal antigen 1/delta-like protein (Jensen et al., 1994). Many of these proteins are implicated in disease and the ability to modulate the activity of these proteins would be of great clinical interest. For example, the EGF receptor is frequently over-expressed in epithelial and pancreatic tumours and those of glial origin, and antibodies directed against this protein have been shown to inhibit the growth of cancer cells (see Baselga et al., (2000) J Clin Oncol 18(4): 904).

Putative consensus sequences for the addition of glycosyl groups to proteins have been identified by comparing the sequences of EGF modules surrounding O-linked fucose and O-linked glucose modification sites (see Harris et al., 1993). In a particular EGF module, the O-linked fucose consensus site is located between the second and third conserved cysteines of the EGF module (C2XXGGS/TC3, where X represents any amino acid). The O-linked glucose consensus site is found between the first and second conserved cysteines (C1XSXPC2). Because the sites are at distinct locations,. both modifications can occur within a single EGF module. Indeed, some proteins (for example, the clotting factors VII and IX) are known to bear both modifications on the same EGF module, demonstrating that the addition of one sugar does not interfere with the addition of the other.

The O-linked Fucose consensus sequence can be found in many EGF repeats. Examples of proteins that contain the putative consensus sequence for modification with O-linked Fucose include Hepatocyte growth factor activator, acrogranin/epithelin, cripto growth factor, Brevican (PCCB), Neurocan (PGCN), Perlecan (PGBM), Versican (PGCV), Multimerin, Fibropellin, Fibrillin, Agrin, Slit, Tyrosine-protein kinase receptor, LDL receptor-related protein (LRP), Cadherin-related tumour suppressor (FAT), Crumbs. Consensus sequences may also be found in a subset of EGF repeats in Notch, Serrate, Delta and in their nematode and vertebrate homologues (see Moloney et al., 1999).

A list of human proteins that contain EGF-like domains is included herewith as Table I. A list of some other proteins that contain the consensus sequence for the addition of O-linked fucose may be found in Table I of Harris and Spellman, 1993 (also see Harris et al., 1993).

A preferred glycosylation target for Fringe and Brainiac is a protein belonging to the Notch family of proteins. Included within the Drosophila Notch family are the receptor Notch, and its ligands Delta and Serrate, all of which contain EGF modules with the glycosylation consensus sequence. In humans, the homologues of these proteins are termed Notch 1-4 (reviewed by Lardelli et al., (1995) Int J Dev Biol 39:769-780), Delta-like 1,2,3 Dlk, and Jagged 1, 2. The Notch and Delta-like genes have been characterised in organisms as diverse as Xenopus, zebrafish, rat, mouse and human, and reveals a striking conservation of gene structure, supporting the relevance of this gene in fundamental control processes of cellular differentiation.

Analysis of the consensus sequences for O-linked fucose and for O-linked glucose addition reveals that human Notch 1 contains 12 O-linked fucose and 17 O-linked glucose sites. Notch-related examples from Drosophila include those sequences identified by the following accession numbers: gb|AAF45848.1| (AE003426) N gene product [D. melanogaster], gb|AAF56276.1| (AE003747) crumbs gene product [D. melanogaster], gb|AAF52472.1| (AE003615) CG9138 gene product [D. melanogaster], gb|AAF51000.1| (AE003576) CG15637 gene product [D. melanogaster], gb|AAF56678.1| (AE003759) Serrate gene product [D. melanogaster], gb|AAF55657.1| (AE003725) Delta gene product [D. melanogaster]and (AAF51333) CG15621 gene product [D. melanogaster]. These proteins and their homologues are examples of proteins that are modified by the glycosyltransferase proteins identified herein.

Other examples of putative Notch-like protein substrates of the glycosyltransferase proteins identified herein, that may be identified from a simple search of publicly-available sequence databases include human Notch homologues such as Notch 2 protein homolog (gi|1082649|pir||A56695[1082649]), Notch 3 ([Homo sapiens] GI|2668592| gb|AAB91371.1|[2668592]); Notch 4 ([Homo sapiens] gi|2072309| gb|AAC32288.1| [2072309]), the Notch protein homolog TAN-1 precursor (human gi|107215|pir||A40043[107215]), Notch-2 ([Mus musculus] gi|975307| gb|AAC52924.1|[975307]), Notch protein homolog (rat gi|112074|pir||S18188[112074]), sequence 16 from U.S. Pat. No. 5,750,652 (gi|3994135 |gb|AAC87563.1| AR007929[3994135]), sequence 34 from U.S. Pat. No. 5,648,464 gi|2488925| gb|AAB77061.1|I56479[2488925]), sequence 37 from U.S. Pat. No. 5,856,441 (gi|5939558|gb|AAE00833.1|[5939558]) and sequence 36 from U.S. Pat. No. 5,856,441 gi|5939557|gb|AAE00832.1|[5939557].

Other proteins that are prospective targets for glycosylation with the Fringe and/or Brainiac proteins may be identified by database searching, for example, by searching the Prosite (http://expasy.hcuge.ch/sprot/prosite.html), Prints (http://iupab.leeds.ac.uk/bmb5dp/prints.html) or Profiles (http://ulrec3.unil.ch/software/PFSCAN_form.html) databases for EGF-like module containing proteins or for proteins that contain consensus glycosylation sites, or by using a search tool such as SMART (http://smart.embl-heidelberg.de).

Notch and Notch-related genes have been implicated in tumour formation. For example, three cases of T-cell acute leukaemia have been demonstrated to be accompanied by a chromosomal translocation in this gene and alterations in Notch signalling have also been associated with neoplastic lesions of the human cervix (Zagouras et al., (1995) P.N.A.S. USA 92:6414). Notch 2 and 3 have also been found to map to regions of neoplasia-associated translocation (Larsson et al., (1994) Genomics 24: 253-258). Due to the convergence of signalling mechanisms between Drosophila and mammals, the results of experiments in flies are considered relevant to the study of human receptors in development and disease. For example, mutations in the Jagged gene have recently been found to be responsible for Alagille syndrome, an autosomal dominant disease characterised by five major abnormalities in the liver, heart, face, vertebrae and eye.

The discovery of the mechanism of regulation of Notch proteins enables the design of agents that inhibit this interaction. Accordingly, a further aspect of the present invention provides for the use of a ligand of a Fringe protein or a ligand of a Brainiac protein as a glycosyltransferase modulator. Preferably, said ligand acts as an inhibitor of the glycosyltransferase activity of a Fringe protein or a Brainiac protein. Such an inhibitor may act on a protein containing one or more EGF-like modules, such as Notch.

Any suitable ligand that modulates the ability of Fringe or Brainiac to transfer a glycosyl group onto a Notch protein is suitable for use in this aspect of the invention. Such ligands may be large molecules such as proteins, or protein fragments, or may, for example, be small molecule drugs or bioactive peptides. The ligands of this aspect of the invention may act as inhibitors or as activators of glycosyltransferase activity.

Preferably, the ligands act as inhibitors. Inhibitors of glycosyltransferases are already available in the art and include inhibitory sugar-pyranoside derivatives such as alpha-D-galactopyranoside; beta-D-galactopyranosyl-; alpha-D-galactopyranoside; beta-D-galactopyranosyl-; beta-D-glucopyranoside; alpha-D-glucopyranoside; beta-D-glucopyranoside; and beta-D-Xylopyranoside.

Advantageously, ligands for use in the present invention should be specific in their action on either or both of Fringe and Brainiac. By “specific” is meant that the ligands bind with high affinity to Fringe or to Brainiac, respectively, but do not bind with any significant degree of affinity to unrelated biological molecules. By high affinity is meant that the ligands bind to the protein with a dissociation constant of at least 10⁻⁶M, preferably, 10⁻⁸M, more preferably 10⁻¹⁰M or less.

The ligand may function to prevent or enhance glycosylation of an EGF-like module containing protein, such as those proteins described above, particularly a protein in the Notch family. The ligand may act by binding to the active site of the Fringe or Brainiac protein, so preventing the protein from carrying out its catalytic function, or to another site on this or another protein so as to improve glycosyltransferase activity.

It should also be possible to modulate the glycosylation of an EGF-like module containing protein by acting directly on the substrate of the Fringe or Brainiac proteins. For example, such a ligand may bind to a consensus site for the addition of an O-linked Fucose residue in an EGF-like module. Accordingly, a further aspect of the present invention provides the use of a ligand of an EGF-like module as a glycosyltransferase inhibitor. The ligands for use in this aspect of the invention should ideally bind to the EGF-like module in the region of the consensus glycosylation site, such that glycosylation of this site is prevented. Again, the ligands for use in this aspect of the invention should ideally bind with high affinity to the target EGF-like module, as described above. Furthermore, such a ligand should be specific for this module. In a particularly preferred embodiment of this aspect of the invention, the ligand should bind specifically and with high affinity to a protein in the Notch family of proteins.

Another possibility included within the present invention is to use a ligand that alters the specificity of the glycosyltransferase reaction carried out by Fringe or Brainiac, for example, by causing the protein to add the incorrect sugar to a site on an EGF module would alter the biological effect of the glycosylation, perhaps preventing chain elongation and other downstream events.

The effect of the ligands described above in modulating glycosylation of the EGF-like module containing protein may include affecting the binding of an effector protein to the EGF-module containing protein such as a Notch protein. By “effector molecule” is meant a ligand of the EGF-module containing protein that has a role in the protein's biological function.

The identities of such ligands will be clear to those of skill in the art. For example, the EGF/TGF-alpha/heregulin proteins are EGF module-containing ligands for receptors that also contain EGF modules. Delta and Serrate are EGF-module containing ligands for Notch. In the case of Notch, the ligands Delta, Serrate, the Delta-like family, and the Jagged family are important to the biological function of the Notch protein. It is hypothesised herein that O-linked fucosylation at consensus sites within the EGF-like modules affects the binding of Delta to Notch. An increase in the glycosylation state of the protein is thought to make the protein more sensitive to the Delta ligand, most likely by allowing Delta to bind to the Notch protein more effectively.

The invention also includes a method of screening for a ligand of a Fringe protein or of a Brainiac protein, or for a ligand of a substrate for a Fringe or Brainiac protein. Such a method may comprise the steps of

a) contacting a candidate ligand with said Fringe or Brainiac protein; and

b) testing the effect of the ligand on the glycosyltransferase activity of said Fringe or Brainiac protein.

A related method may be used to screen for a ligand of a substrate for a Fringe or Brainiac protein, for example, including the steps of:

a) contacting a candidate ligand with said substrate in the presence of a Fringe or a Brainiac protein; and

b) testing the effect of the ligand on the glycosylation state of said substrate.

For example, libraries of compounds may be screened, using any one of a variety of screening techniques. Such compounds may activate (agonise) or inhibit (antagonise) the level of expression of the Fringe or Brainiac gene or may modulate the activity of either or both of these polypeptides, or their functional equivalents. Suitable ligands of this nature may be isolated from, for example, cells, cell-free preparations, chemical libraries or natural product mixtures. Examples of types of ligand compounds as defined herein include natural or modified substrates, small organic molecules, peptides, polypeptides, antibodies, enzymes, receptors, structural or functional mimetics. For a suitable review of such screening techniques, see Coligan et al., Current Protocols in Immunology 1(2):Chapter 5 (1991).

The Fringe or Brainiac polypeptide, or functional equivalent thereof, that is employed in such a screening technique may be free in solution, affixed to a solid support, borne on a cell surface or located intracellularly. In general, such screening procedures may involve using appropriate cells or cell membranes that express the appropriate polypeptide that are contacted with a test ligand compound to observe binding, or stimulation or inhibition of a functional response, such as the glycosyltransferase activity of the protein. When testing for ligands that are effective to inhibit the glycosylation of a Fringe or Brainiac substrate (such as an EGF module), the functional response may be the presence absence or alteration of substrate modification. The functional response of the cells contacted with the test compound is then compared with control cells that were not contacted with the test compound.

Alternatively, simple binding assays may be used, in which the adherence of a test ligand compound to a surface bearing the Fringe or Brainiac polypeptide is detected by means of a label directly or indirectly associated with the test compound or in an assay involving competition with a labelled competitor. In another embodiment, competitive drug screening assays may be used, in which neutralising antibodies that are capable of binding the polypeptide specifically compete with a test ligand compound for binding. In this manner, the antibodies can be used to detect the presence of any test compound that possesses specific binding affinity for the Fringe or Brainiac polypeptide.

Assays may also be designed to detect the effect of added test compounds on the production of mRNA encoding the Fringe or Brainiac polypeptide in cells. For example, an ELISA may be constructed that measures secreted or cell-associated levels of polypeptide using monoclonal or polyclonal antibodies by standard methods known in the art, and this can be used to search for compounds that may inhibit or enhance the production of the polypeptide from suitably manipulated cells or tissues. The formation of binding complexes between the polypeptide and the compound being tested may then be measured.

Another technique for drug screening which may be used provides for high throughput screening of compounds having suitable binding affinity to the polypeptide of interest (for example, see International patent application WO84/03564). In this method, large numbers of different small test compounds are synthesised on a solid substrate, which may then be reacted with a polypeptide and washed. One way of immobilising the polypeptide is to use non-neutralising antibodies. Bound polypeptide may then be detected using methods that are well known in the art. Purified polypeptide can also be coated directly onto plates for use in the aforementioned drug screening techniques.

A still further technique involves the screening of the transcriptomes or proteomes of cells or organisms in which the level of expression or the level of activity of either or both of Fringe and Brainiac has been modified. This aspect of the invention provides a method for the identification of a gene that is implicated in a disease or physiological condition in which Fringe or Brainiac function plays a role, said method comprising the steps of:

a) comparing:

i) the transcriptome or proteome of a first cell type; with

ii) the transcriptome or proteome of a second cell type in which the expression or activity of one or both Fringe and Brainiac proteins is altered in comparison to the first cell type; and

b) identifying as the gene implicated in the disease or condition:

i) a gene that is differentially regulated in the two cell types; or

ii) a gene encoding a protein whose level of glycosylation differs between the two cell types.

By “transcriptome” is meant the exact set of transcripts that are expressed in a cell. For example, nucleic acid arrays provide a useful tool for the study of transcriptome variation between different tissue types. These tools facilitate the evaluation of variations in DNA or RNA sequences and of variations in expression levels from tissue samples and allow the identification and genotyping of mutations and polymorphisms in these sequences (see, for example, Schena et al., 1995 (Science 270: 467-470) and Fodor et al., 1991 (Science 251, 767-773). Other techniques that are suitable for the analysis of the transcriptome of a specific cell type include serial analysis of gene expression (SAGE; Velculescu et al., Science (1995) 270; 484-487), Selective amplification via biotin- and restriction-mediated enrichment (SABRE) (Lavery et al, (1997), PNAS USA 94: p6831-6836); Differential display (for example, indexing differential display reverse transcriptase polymerase chain reaction (DDRT-PCR; Mahadeva et al. (1998) J. Mol.Biol. 284, 1391-1398); representational difference analysis (RDA) (Hubank (1999) Methods in Enzymology 303: 325-349); differential screening of cDNA libraries (see Sagerstrom et aL (1997) Annu. Rev. Biochem. 66: 751-783); “Advanced Molecular Biology”, R. M. Twyman (1998) Bios Scientific Publishers, Oxford; “Nucleic Acid Hybridization”, M. L. M. Anderson (1999) Bios Scientific Publishers, Oxford); Northern blotting; RNAse protection assays; S1-nuclease protection assays; RT-PCR; real time RT-PCR (Taq-man); EST sequencing; massively parallel signature sequencing (MPSS); and sequencing by hybridisation (SBH) (see Drmanac R. et al (1999), Methods in Enzymology 303:165-178). Many of these techniques are reviewed in “Comparative gene-expression analysis” Trends Biotechnol. February 1999;17(2):73-8.

Such studies may alternatively, or in addition, involve proteomics analyses. The use of two dimensional SDS-PAGE gels in combination with amino acid sequencing by mass spectrometry is currently the most widely-used technique in this field (see “Proteomics to study genes and genomes” Akhilesh Pandey and Matthias Mann, (2000), Nature 405: 837-846). Additionally, the recent developments in the field of protein and antibody arrays now allow the simultaneous detection of a large number of proteins. For example, low-density protein arrays on filter membranes, such as the universal protein array system (Ge, (2000) Nucleic Acids Res. 28(2), e3) allow imaging of arrayed antigens using standard ELISA techniques and a scanning charge-coupled device (CCD) detector.

Modified cells and organisms may be used to identify drug targets downstream of Fringe and Brainiac action. For example, a transgenic animal or transfected cell population might be created in which Fringe or Brainiac has been knocked out, misexpressed (for example, by targeted or random mutagenesis) or overexpressed. Studies performed on such cells or organisms may reveal genes and proteins that act downstream in the same metabolic or developmental pathway as Fringe or Brainiac, that are thus potential targets for Fringe or Brainiac function. Furthermore, the protein/lipid glycosylation status of the animal's cells may be assessed to identify potential targets for Fringe or Brainiac function that could then be studied further as candidate drug targets.

A further aspect of the invention provides for the use of a Fringe protein or a Brainiac protein, or a fragment, or functional equivalent of a Fringe protein or a Brainiac protein, or of a ligand as described in any one of the embodiments of the invention described above, in the manufacture of a medicament for the treatment of a disease caused by an EGF-like module containing protein.

Such diseases include T cell leukaemia, breast cancer, stroke, dementia, cerebral autosomal dominant arteriopathy with subcortical infarcts, leukoencephalopathy and Alagille syndrome. In a preferred aspect of this embodiment of the invention, diseases suitable for treatment may be caused by a defect in the Notch signalling pathway.

According to a further aspect of the invention, there is provided a method of treating a disease caused by an EGF-like module containing protein comprising administering to a patient a Fringe protein or a Brainiac protein, or a fragment, or functional equivalent of a Fringe protein or a Brainiac protein, or of a ligand as described in any one of the embodiments of the invention described above.

A further aspect of the present invention provides a Fringe protein or a Brainiac protein, or a fragment, or functional equivalent of a Fringe protein or a Brainiac protein, for use as a glycosyltransferase.

According to a still further aspect of the invention, there is provided a method of transferring a N-acetylglucosamine moiety onto a fucose or a mannose substrate, whether free or attached to a lipid, carbohydrate or protein, comprising the step of incubating a Fringe protein or a Brainiac protein, or a fragment, or functional equivalent of a Fringe protein or a Brainiac protein with its substrate. Preferably, the transfer of the N-acetylglucosamine moiety is onto a protein, such as a EGF-module containing protein as described above.

Various aspects and embodiments of the present invention will now be described in more detail by way of example. It will be appreciated that modification of detail may be made without departing from the scope of the invention.

### BRIEF DESCRIPTION OF THE FIGURES

FIG. 1: Alignment of members of the Fringe and Brainiac protein families.

FIG. 2: Phylogenetic tree showing members of the Fringe and Brainiac protein families.

FIG. 3: Fringe increases binding of Delta to Notch

a) Schematic representation of Delta and the secreted Delta-alkaline phosphatase fusion protein used in binding assays.

b) Upper panel: Delta-AP binding to control and transfected S2 cells. Lower panel: Immunoblot of cell extracts prepared in parallel to those used in the binding assay and probed with anti-Notch. Tubulin levels were assayed to control for loading of total cellular protein (not shown).

FIG. 4: Golgi-tethered Fringe increases binding of Delta to Notch

a) Schematic representation of wild-type and Golgi-tethered Fringe (Fng Ga1NT3).

b) Immunoblot of myc-tagged proteins immunoprecipitated from total cell lysates (cells) and from medium conditioned by cells transfected with Fringe-wt-myc or Fringe-GalNT3-myc and from control cells transfected with empty vector.

c) Immunofluorescent labeling of SL2 cells transfected to express Golgi-tethered Fringe and labelled with antibody. Golgi-tethered Fringe (Fng GT-myc) colocalizes with the Golgi marker in the transfected cells.

d) Delta-AP binding to Notch-expressing cells is stimulated by coexpression of Fringe-myc or Fringe-GalNT3-myc. Replicate experiments are shown. Inmunoblots of protein extracts from the transfected target cells probed with anti-Notch or anti-myc are shown below. Tubulin levels were assayed to control for loading of total cellular protein (not shown).

(e), (f), (g) Wing imaginal discs labelled to visualise Wingless protein (left panel) and the myc epitope tag (right panel). (e) patched^{GAL4}/+wing disc. Wingless is expressed in a continuous stripe along the DV boundary. (f) patched^{GAL4}/UAS-Fringe-GalNT3-myc. Golgi-tethered Fringe-GalNT3-myc expression in the patched^{GAL4} stripe is shown in the right-hand panel. The endogenous Wingless stripe is interrupted where the Golgi-tethered Fringe stripe crosses the DV boundary (left panel). At the anterior-posterior compartment boundary the Golgi-tethered Fringe stripe abuts the endogenous Wingless stripe and induces ectopic expression of Wingless in the ventral compartment. This pattern is identical to that reported for ectopic expression of wild-type (secreted) Fringe shown in panel (g) for the Golgi-tethered Fringe D: dorsal compartment. V ventral compartment. Posterior is to the right.

FIG. 5: Fringe has glycosyltransferase activity

a) Delta-AP binding to control and Notch transfected S2 cells. Lower panels: Immunoblots of duplicate cell lysates probed with anti-Notch, with anti-Myc and with anti-Tubulin as a control for total cellular protein in the extracts.

b) Glycosyltransferase activity in microsomal fractions from cells expressing Fringe-wt-myc or Fringe-NNN-myc.

FIG. 6: Secreted Notch produced by Fringe-GalNT3 expressing cells binds Delta expressing cells

a) Schematic representation of Notch, Notch-CD2 and the secreted Notch-AP fusion protein used in binding assays. Notch-CD2 consists of the EGF repeats of Notch fused to the heterologous transmembrane protein CD2. Notch-AP consists of the same region of Notch expressed as a secreted AP fusion protein. Asterisks indicate EGF repeats that contain a perfect consensus sequence for O-linked Fucose modification (CxxGGS/TC). TM indicates the membrane-spanning domain. Gray circles indicate ankyrin repeats in the cytoplasmic tail of Notch.

b) Delta-AP binding to cells expressing Notch-CD2. Lower panel: immunoblot probed with anti-CD2 to visualise expression of the Notch-CD2 fusion protein. Expression is comparable in the presence or absence of Fringe-CD2. Mouse Notch1 is cleaved in the extracellular domain after the third NL repeat by a furin protease. This region of the protein has been replaced by CD2 sequences in Notch-CD2. Notch-CD2 does not appear to be proteolytically processed and migrates at approximately 200 kD.

c) Binding of Notch-AP produced by S2 cells (gray bars) and Notch-AP produced by S2 cells also expressing golgi-tethered Fringe-GalNT3-myc (black bars) to cells expressing Delta or to control cells transfected with empty vector. The level of AP activity in the conditioned media was normalised for the binding assay. We were unable to detect measurable binding of N-AP to Serrate-expressing cells (not shown).

FIG. 7: Glycosylation of Notch by Fringe in vitro

a) Aligned amino acid sequence of the first three EGF repeats of Notch. Conserved residues and the consensus sequence for O-fucosylation are indicated (arrow).

b) Glycosyltransferase activity measured in microsomal fractions from cells expressing Fringe-wt-myc or Fringe-NNN-myc. Enzyme activity was measured by transfer of ¹⁴C-labelled UDP-GlcNAc onto Notch-EGF3-His. Average results from two experiments are shown.

c) SDS-PAGE of samples from (b) run on a 15% acrylamide gel. Upper panel: Coomassie blue stained gel. The Notch-EGF-3 protein is indicated. The samples contained a relatively large amount of protein from the microsome fractions, which bound non-specifically to the beads. Lower panel: ¹⁴C-UDP-GlcNAc-labeled proteins visualised by autoradiography. Only the N-EGF3 protein was labelled.

FIG. 8: Brainiac activity towards glycosphingolipids purified from insect cells. Microsomal fractions (1.5 mg) of insect cells transfected with pVL-brainiac and pVL-fringe were used as the enzyme source. Reactions contained 10 μg of purified glycolipids and 300 μM of UDP-¹⁴C-N-acetylglucosamine (8200 cpm/nmol). Products were purified on C18-silica cartridges, spotted on HPTLC-plates, developed in 60:35:8 (chloroform:methanol:water) and exposed for autoradiography.

### EXAMPLES

### Methods

Constructs: The metallothionine-inducible Notch expression construct in pRmHa3 is described in Fehon et al., 1990.

Notch-AP was constructed by cloning sequences encoding amino acids 1-1467 of Notch in frame with human placental Alkaline Phosphatase from pcDNA3-AP (Bergemann et al., 1995). The fusion junction is located at a unique BspEI site between the last EGF repeat and the first NL repeat. The same Notch fragment was used to make Notch-CD2 and were linked in frame to rat CD2 at residue 2 (following the signal sequence, as described in Strigini and Cohen, 1997).

Delta-AP: A BglII site was introduced by PCR following residue N592 and sequences encoding aa 1-592 of Delta were fused in frame with alkaline phosphatase at the BglII site of pcDNA3-AP.

Fringe-myc: A single myc-epitope tag was introduced at the C-terminus of Fringe by PCR. Amino acids added were EFEQKLISEEDL. Fringe-myc was cloned into pRmHa3 for expression in S2 cells and into pUAST for GAL4-regulated expression in Drosophila.

Fringe-NNN-myc: Amino acids D236, 237 and 238 of Fringe-myc were converted to asparagine residues by PCR.

Fringe-Ga1NT3-myc: Fragments encoding the first 121 amino acids of GalNAc-T3 and aa 40-424 of Fringe-myc were amplified by PCR using oligonucleotides which produce a 15 bp overlapping sequence at the fusion junction. The first two PCR products were used as template to amplify the full length fusion.

Brainiac-full: The full coding region of Brainiac was prepared by PCR using genomic DNA.

Cell culture and Binding assays: cDNAs for expression in Schneider S2 cells were cloned into pRmHa3. Cells were transiently transfected by the CaPO₄ method using 6-8 μg of DNA per well in 6 well plates. Expression was induced by addition of 0.7 mM CuSO₄ for 2 days. Conditioned medium was collected from Notch-AP and Delta-AP transfected cells 2 days after induction. AP activity was adjusted to normalise activity levels of Notch-AP or Delta-AP expressed with or without Fringe.

Binding was performed as described in Cheng and Flanagan, 1994. In brief, AP-containing supernatants were supplemented with 0.1% NaN₃, and incubated 90 min at room temperature. Cells were washed 5 times in HBSS containing 0.05% BSA and 0.1% azide, and lysed in 10 mM Tris pH 8, 1% Triton-X100. Endogenous AP was inactivated by heat treatment for 10 min 60° C. and the lysates clarified by centrifugation. AP activity was measured in 1M Diethanolamine, 1 mM MgCl₂, 5 mM para-nitrophenyl phosphate. Bound AP activity was quantified in 96 well plates using a microplate reader and Micromanager software (BioRad). An additional replicate of each transfection was prepared for immunoblot analysis. Lysates were prepared separately for Immunoblot analysis to allow inclusion of protease inhibitors which are not used in the binding assay.

### Glycosyltransferase Assays

Fringe

Fringe-Myc and Fringe-NNN-myc were cloned into baculovirus vector pVL1393 (Pharmingen) and expressed in High Five™ cells. Microsomal fractions were prepared by hypotonic lysis ultracentrifugation and solubilised 1:2 (vol/vol) in 20 mM Cacodylate pH 6.5, 1% Triton-CF54, 5 MM MnCl₂ containing Leupeptin and Aprotinin as described previously (Amado et al., 1998). For glycosyltransferase assays, 5 μl of this suspension were added to a total of 50 μl reaction mixture containing 25 mM Cacodylate pH 6.5, 0.25% Triton-CF54, 5 mM MnCl₂, 500 mM free sugar, 100 μM UDP-¹⁴C-sugar (1280-2000 cpm/nmol). Reactions were incubated at 37° C. for 45-60 minutes, followed by Dowex-1 anion exchange chromatography and scintillation counting of 50% of the flow-through. The hydrophobic fucosyl-aglycans, , α-L-Fuc-1-p-Nph and β-L-Fuc-1-thio-p-Nph did not act as acceptors for Fringe in this assay (not shown), consequently we were unable to characterise the type of linkage between GlcNAc and Fuc catalysed by Fringe.

Brainiac

Brainiac-full was cloned into baculovirus vector pVL1393 (Pharmingen) and expressed in High Five cells. Microsomal fractions were prepared as described above for Fringe but with 0.5% N-octylglucoside as Triton X-100 and related detergents destroyed the activity. Initial analysis of activity was done with five monosaccharides and four donor sugar nucleotides as described in FIG. 5 for Fringe. Assays with the acceptors listed in Table 1 were performed as described for Fringe except no additional detergent was added. Reactions with glycolipid substrates were performed in the presence of 0.5% N-octylglucoside in 50 ul reaction volume containing 10 μg glycolipid.

Glycolipids were obtained from Sigma or purified from High Five insect cells grown in serum free medium. The High Five glycolipids migrating as ceramide dihexoside (CDH) and ceramide trihexoside (CTH) were isolated from the lower phase of a Folch partition, and purified by HPLC. Analysis by ¹H-NMR showed the structures to be Manβ1-4Glcβ1-Cer and Galβ1-4Manβ1-4Glcβ1-Cer, respectively. Analysis of activity of Brainiac with glycolipid acceptors was performed essentially as described in Amado et al., 1998. For analysis of the linkage formed by Brainiac, reactions with UDP-GlcNAc in combination with Manβ1-4Glcβ1-Cer or Manβ1-MeUmb were run to completion and the product purified by C18 SepPack chromatography. Analysis by ¹H-NMR showed that the structure formed was GlcNAcβ1-3Manβ1-4Glcβ1-Cer and GlcNAcβ1-3Manβ1-MeUmb, respectively.

Immunoprecipitation and immunoblots were done as described by Bruckner et al., 1999, 1997. Cells were lysed in 50 mM Tris pH 7.5, 1% TritonX100, 120 mM NaCl, 30 mM NaF, containing protease inhibitors. Antibodies for immunoprecipitation and western blots include mouse monoclonal anti-Myc (9E10), rabbit anti-Myc (Santa Cruz Biotechnology), mouse monoclonal anti-Notch 9C6 (Fehon et al. 1990). Protein bands were visualised with peroxidase conjugated secondary antibodies and enhanced chemiluminescense.

### Example 1

Effect of Fringe on Notch-Delta Binding

In the developing Drosophila wing, asymmetric activation of Notch by the dorsally-expressed ligand Serrate and the ventrally-expressed ligand Delta is required to induce Wingless and Vestigial expression and establish a signalling centre at the dorsal-ventral boundary. Fringe is known to be expressed in dorsal cells and contributes to making them more sensitive to Delta and less sensitive to Serrate.

One means by which Fringe might change the cells' sensitivity to Notch ligands is by directly modulating ligand-receptor interaction. Alternatively, Fringe might act directly to influence cellular signalling responses to a given level of ligand binding.

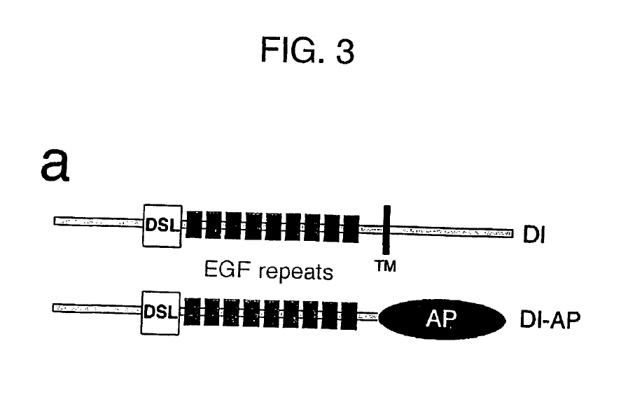

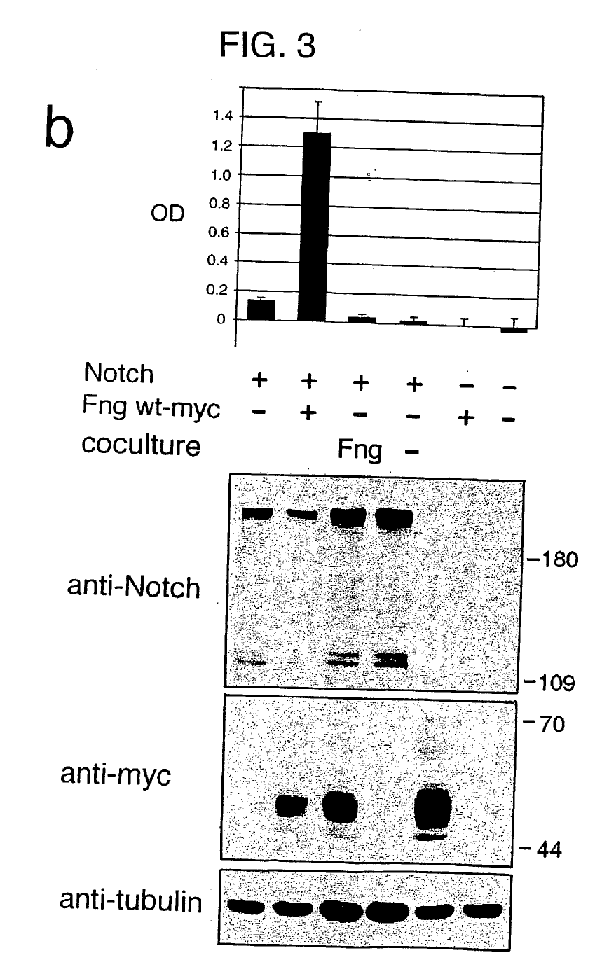

To distinguish between these possibilities, we measured the effect of Fringe on Notch-Delta binding. We expressed the extracellular domain of Delta as a secreted Alkaline Phosphatase fusion-protein for use in a ligand binding assay (Delta-AP; FIG. 3a).

To measure interaction of Delta-AP with transiently transfected S2 cells, bound AP activity was quantified in an enzymatic colour reaction. Cells were transfected with constructs to direct expression of Notch or Fringe-myc as indicated in the Figure. Cells transfected with empty vector were used as a control. “Coculture” indicates that cells transfected with Notch were grown as a mixed culture with cells independently transfected to express Fringe-myc or with cells transfected with empty vector. AP activity is shown in mOD units/minute in cell extracts (replicate experiments are shown in this figure). In this experiment Fringe stimulated binding by over 50 fold. The Delta-AP binding assay does not appear to be as sensitive as the immunoblot assay for binding endogenous secreted Delta in that it does not detect significant binding of Delta-AP to cells that express Notch alone. However, the assay allows measurement of Fringe-dependent stimulation of Delta-AP binding.

Expression levels of the transfected proteins were monitored by immunoblot analysis in parallel to the binding assays. S2 cells expressing Notch were found to bind Delta-AP at a level that is not distinguishable from control cells (FIG. 3b). In contrast, co-expression of Notch and Fringe resulted in large increase in Delta-AP binding, which was not seen in cells expressing Fringe alone (FIG. 3b). The level of Notch expression and the proteolytic maturation required for formation of a functional receptor was not increased by co-expression of Fringe. This suggests that Fringe activity may increase the ability of Notch-expressing cells to bind Delta.

### Example 2

Effect of Secreted Fringe Protein

Although Fringe and its vertebrate homologues can be found as secreted proteins, genetic analysis has suggested that Fringe acts cell autonomously in the wing disc. These apparently contradictory observations raise the possibility that secreted Fringe might not be able to affect Notch-Delta binding.

To test this we compared Delta-AP binding to cells in which Notch and Fringe were co-expressed with binding to Notch-expressing cells that were co-cultured with Fringe-expressing cells for two days prior to the binding assay FIG. 3b).

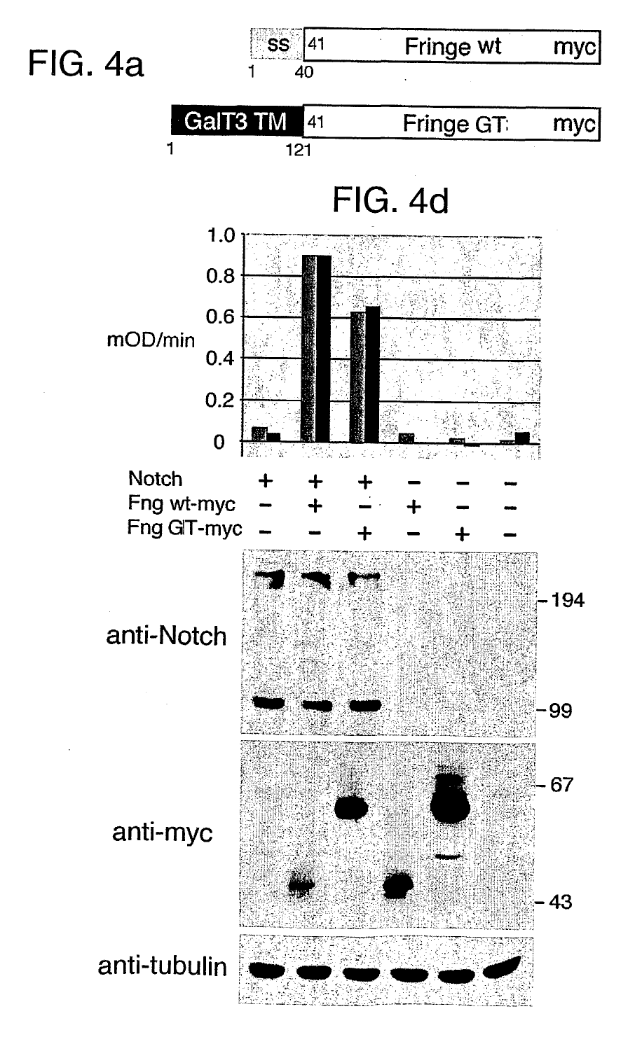

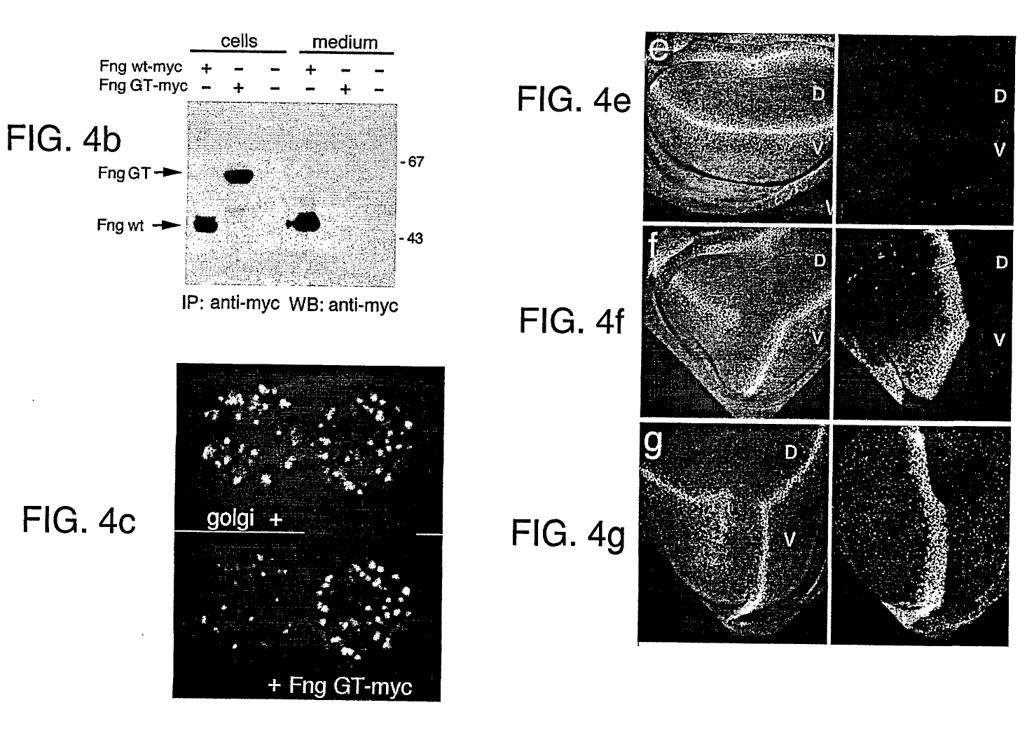

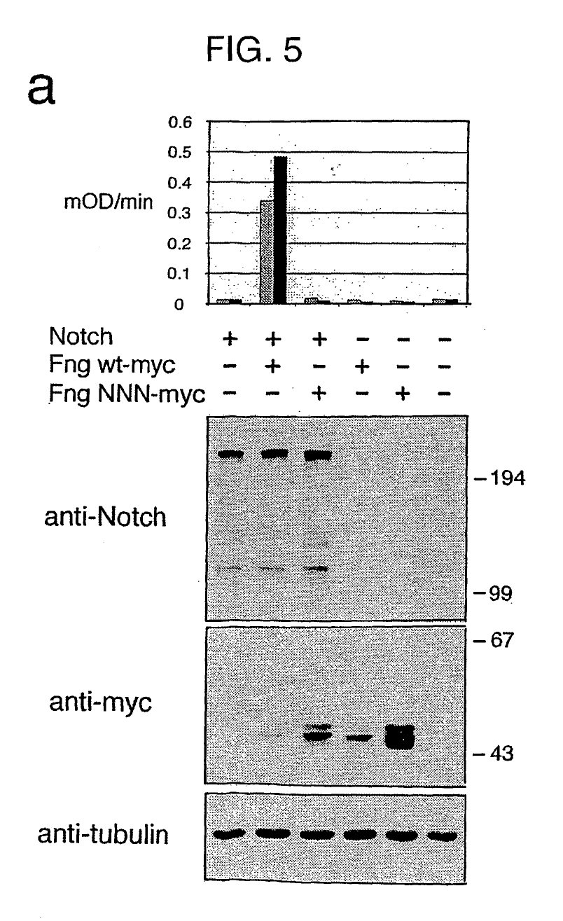

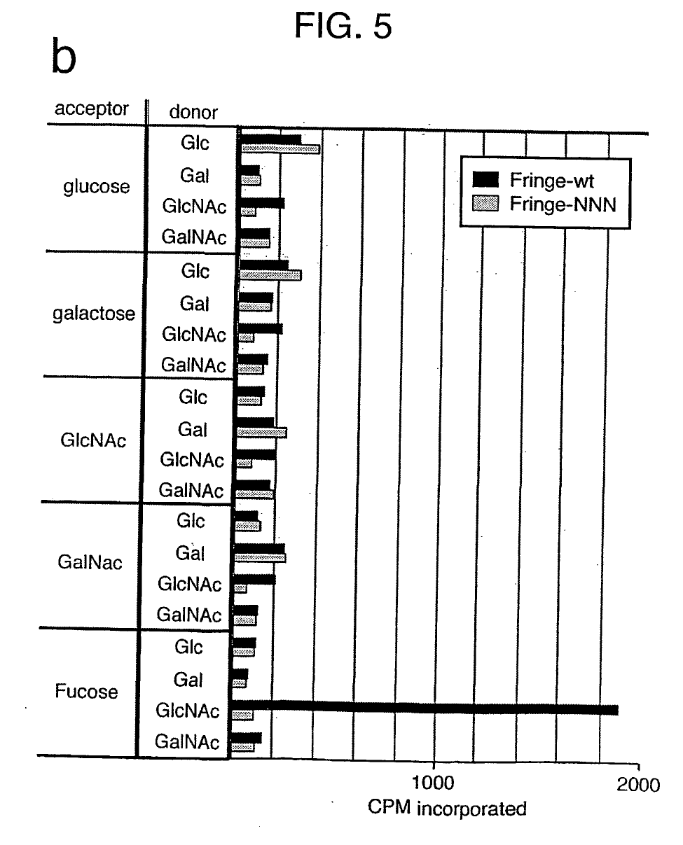

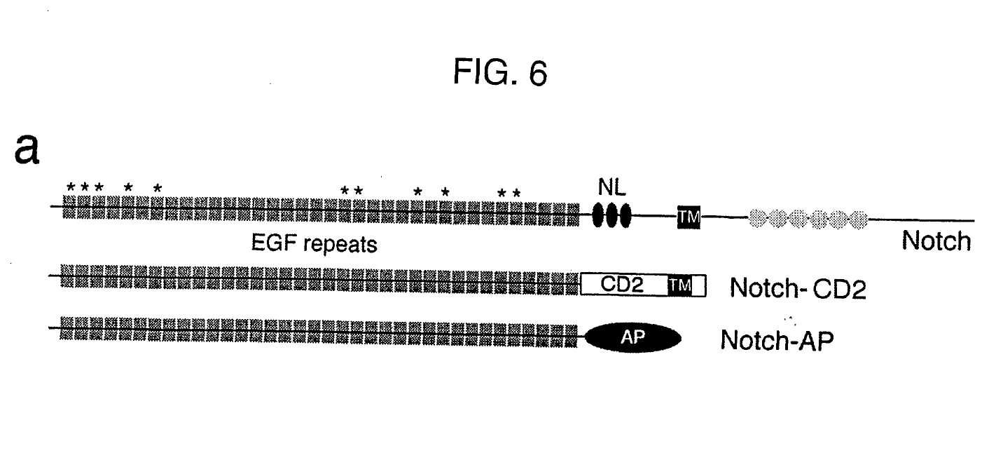

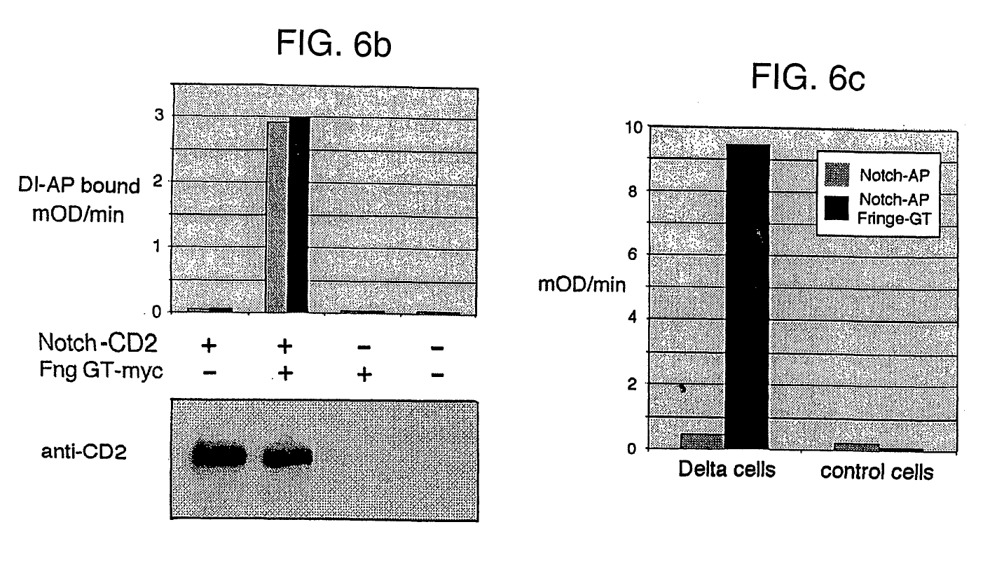

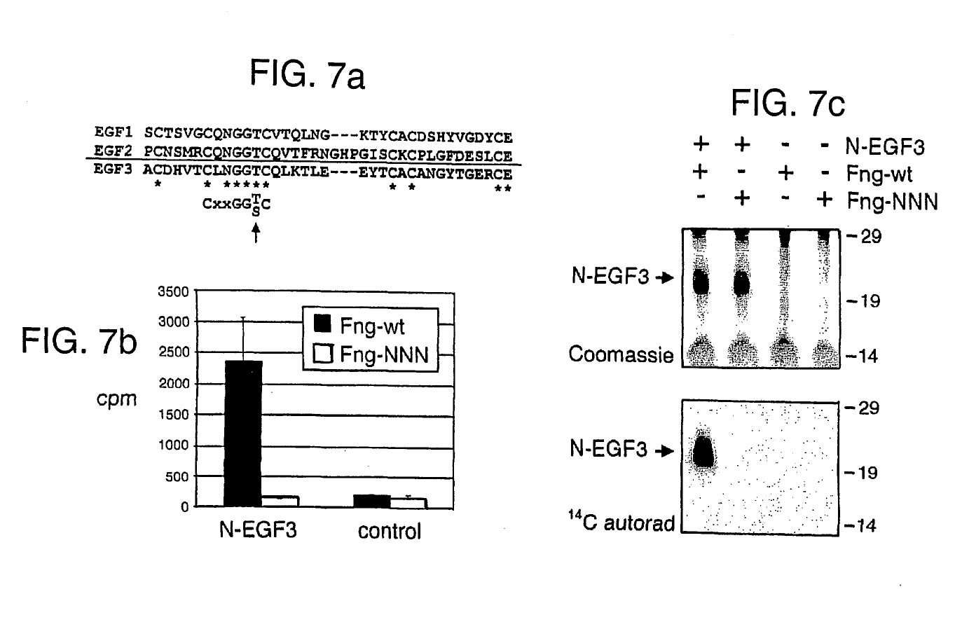

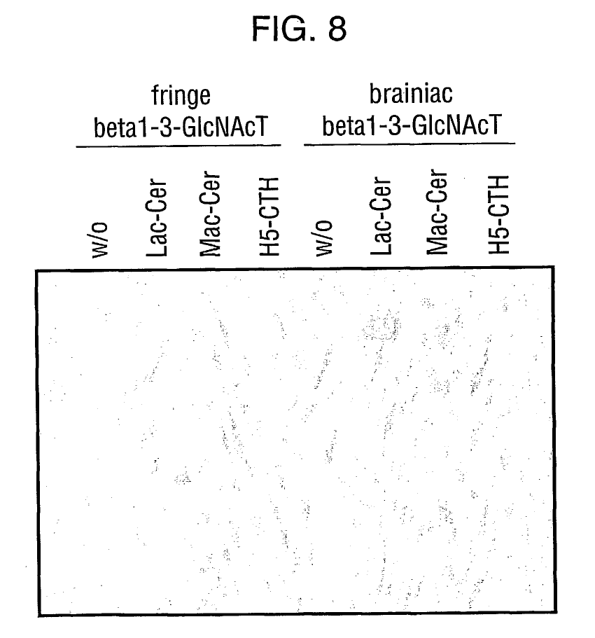

Delta-AP bound at background level to co-cultured cells, comparable to the level obtained with cells transfected with Notch alone or with cells transfected with empty vector. A substantial amount of Fringe protein can be detected in the medium of transfected S2 cells (see FIG. 4b). However, Fringe does not appear to be able to influence the ability of Notch to bind Delta when provided as an extracellular protein, but does act when coexpressed with Notch.

### Example 3

Fringe has glycosyltransferase activity

The requirement for co-expression of Fringe and Notch could be explained if Fringe exerts its activity within the Notch-expressing cell. The similarity that Fringe and Brainiac show to various bacterial glycosyltransferase enzymes has been reported previously (Yuan et al., 1997), and several mammalian glycosyltransferases that show regions of homology to Brainiac have been functionally characterized.

To test the possibility that Fringe functions as a glycosyltransferase enzyme, we prepared a Golgi-tethered version of Fringe in which the first 40 amino acids (including the predicted signal peptide) were replaced by the first 121 amino acids of the Golgi-resident glycosyltransferase enzyme GalNAc-T3. Both proteins carry a C-terminal myc epitope-tag (FIG. 4a; Bennett et al., 1996; Röttger et al., 1998). This rationale relies on the assumption that if Fringe acts as a glycosyltransferase, it will do so in the Golgi. The Fringe-GT fusion protein that was produced includes the transmembrane domain, which functions as a Golgi-retention signal for GaINAc-T3 (Nilsson and Warren, 1994).

Immunoprecipitation of both proteins from transfected S2 cells showed that Fringe-GT was expressed comparably to wild-type Fringe, but was not secreted at detectable levels (FIG. 4b). This confirmed that the transmembrane tether provided by Ga1NAc-T3 is effective in S2 cells. In binding experiments, co-expression of Fringe-GT was sufficient to stimulate Delta-AP binding to Notch almost is effectively as wild-type Fringe (FIG. 4c). This suggests that Fringe-GT has comparable activity to wild-type Fringe. Fringe-GT was also found to be functional in vivo, despite not being secreted.

When expressed under patched^{GAL4}, control Fringe-GT had no effect in the dorsal compartment where endogenous Fringe is expressed, but interrupted the endogenous Wingless stripe at the DV boundary and induced ectopic expression of Wingless in the ventral compartment (FIG. 4d). These effects precisely match those caused by expression of wild-type Fringe (not shown; see Panin et al., 1997; Kim et al., 1995). These observations suggest that a Golgi-resident form of Fringe has full biological activity.

Many Golgi glycosyltransferase enzymes are also found as secreted soluble enzymes, though the function of the secreted forms is unknown. Thus the fact that Fringe proteins are secreted may not be of functional significance to their roles as modifiers of Notch activity (Wu et al., 1996).

### Example 4

Mutation of Fringe abolishes glycosyltransferase activity

A D-x-D sequence motif found in many glycosyltransferases is required for catalytic activity (Yuan et al., 1997; Hagen et al., 1999) and appear to be directly involved in coordination of a divalent metal ion in the binding of the donor nucleotide sugar (Breton and Imberty, 1999; Gastinel et al., 1999).

In Fringe, the D-x-D motif corresponds to residues D236-238. If Fringe acts as a glycosytransferase, replacing residues D236-238 with Asparagine (Fringe-NNN) should destroy enzymatic activity while having a minimal effect on overall protein structure. We therefore tested the Fringe-NNN mutant in the S2 Delta-AP binding assay.

Cells were transfected to express Notch, wild-type Fringe-wt-myc, or mutant Fringe-NNN-myc as indicated. Cells transfected with empty vector were used as a control. Fringe-NNN-Myc has no activity in the binding assay, despite being expressed at higher levels than Fringe-wt-Myc. Consistent with the possibility that Fringe activity requires the putative catalytic residues, we observe that co-expression of Fringe-NNN with Notch did not increase Delta-AP binding above background levels (FIG. 5a). Furthermore, ectopic expression of Fringe-NNN under patched^{GAL4} control did not cause Notch activation in the ventral compartment in the wing imaginal disc (not shown). These observations suggest that Fringe-NNN is inactive in vivo.

To ask whether Fringe has intrinsic glycosyltransferase activity, Fringe-wt and Fring-NNN were produced by baculovirus-infection of insect cells. Microsomal fractions enriched for Golgi membranes were partially solubilized and assayed for the ability to the expressed proteins to catalyze the transfer of ¹⁴C-labelled UDP-donor sugars onto acceptor sugars. A variety of different donor/acceptor combinations were tested (FIG. 5b).

Enzyme activity was measured by transfer of ¹⁴C-labelled donor sugars (as UDP-conjugates) onto acceptor sugars. In FIG. 5b, average results from two experiments are shown. Donors tested were UDP-Glucose (Glc), UDP-Galactose (Gal), UDP-N-acetyl-Glucosamine (GlcNAc) and N-acetyl-Galactosamine (GalNAc). Acceptors tested were Glucose, Galactose, GlcNAc, GalNAc and Fucose. The highest level of activity was observed with Fringe-wt microsome lysate and the combination of UDP-N-acetylglucosamine (GlcNAc) and Fucose (18-fold over background level observed with Fringe-NNN), suggesting the Fringe has glycosyltransferase activity. Fringe showed no significant activity with other donor-acceptor combinations.

Fucose is commonly found as an unsubstituted terminal sugar residue in N- and O-linked oligosaccharide chains of glycoproteins and in glycosphingolipids of eukaryotic cells. In contrast, addition of O-linked Fucose directly to proteins is a rare type of glycosylation that is found in association with the cysteine-rich consensus sequence C-x-x-G-G-S/T-C (Harris and Spellman, 1993). Elongation of the O-linked Fucose occurs only in a subset of proteins modified by this type of glycosylation. O-linked Fucose may be extended by addition of β1-3 GlcNAc followed by addition of galactose and sialic acid residues.

Remarkably, the O-linked Fucose consensus sequence is found in EGF repeats, including a subset of those in Notch, Serrate, Delta and in their nematode and vertebrate homologues (Moloney et al., 1999, see FIG. 6a). Cells were transfected to express Notch-CD2 or Fringe-GT-myc as indicated in FIG. 6b. Cells transfected with empty vector were used as a control. Our results suggest that Fringe acts by elongating O-linked Fucose residues in the EGF repeats of Notch through addition of β1-3 GlcNAc.

### Example 5

Fringe Acts Via the EGF Repeats of Notch

To ask whether Fringe acts via the EGF repeats of Notch, we expressed the EGF Notch as a fusion protein in which all sequences following the EGF repeats were replaced by heterologous sequences from the transmembrane protein CD2 (FIG. 6a). Cells expressing Notch-CD2 and Golgi-tethered Fringe-GT bound over 50-fold more Delta-AP than cells expressing Notch-CD2 alone or control cells (FIG. 6b). This observation indicates that when taken out of their normal context in the endogenous Notch protein, the EGF repeats are sufficient to mediate binding to Delta.

Under normal circumstances, expression of Notch on the cell surface requires proteolytic cleavage in the extracellular domain by a Furin-like protease (Blaumueller et al., 1997; Logeat et al., 1998). The cleaved extracellular domain remains attached to the transmembrane and intracellular domain to form an active signalling complex (reviewed in Artavanis-Tsakonas et al., 1999). The need for proteolytic processing and any other modifications that may depend on such processing appear to be circumvented in the Notch-CD2 fusion protein.

### Example 6

Production of Notch Expressed as a Soluble AP Fusion Protein

As the EGF repeats of Notch appear to be sufficient to mediate ligand interaction, we reasoned that the corresponding domain of Notch expressed as a soluble AP fusion protein might retain ligand binding activity (FIG. 6a).

Notch-AP was produced by control S2 cells and by S2 cells that also expressed Golgi-tethered Fringe-GT. Binding of the secreted AP-fusion proteins was measured using S2 cells expressing full length Delta as a transmembrane protein. Notch-AP produced in cells expressing Fringe-GT-myc bound to Delta-expressing cells 20-fold more effectively that Notch-AP produced in the absence of Fringe (FIG. 6b). This suggests that the observed binding relies solely on the EGF-repeats of Notch provided as a secreted protein.

To ask whether Fringe acts directly to modify one or more EGF repeats of Notch, we carried out an in vitro glycosylation assay using a short protein consisting of the first three EGF repeats of Notch as the substrate. The first three EGF repeats of Notch contain perfect consensus sequences for the addition of O-linked Fucose (FIG. 7a). The results presented above suggest that Fringe might act by elongating O-linked Fucose through addition of GlcNAc. The Notch-EGF3 protein was expressed in SL2 cells with a C-terminal His-tag to permit purification of the secreted protein. Equal amounts of Notch-EGF3 were incubated with ¹⁴C-labeled UDP-GlcNAc and microsomal lysates from cells expressing Fringe-wt or Fringe-NNN. At least 16-fold more labelled GlcNAc was incorporated into Notch-EGF3 by wild type Fringe than by the mutant form Fringe-NNN (FIG. 7b, c). We conclude that Fringe can act directly to modify the EGF repeats of Notch.

### Example 7

Brainiac encodes β1,3N-acetylglucosaminyltransferase

Initial analysis of putative glycosyltransferase activity of Brainiac expressed as a full coding construct in High Five cells using a panel of monosaccharides and four UDP sugar nucleotides showed increased activity compared to control cells only with UDP-GlcNAc similar to Fringe. D-Mannose was the best acceptor although L-Fucose also served as a poor substrate. A more detailed analysis with a panel of mono- and disaccharides attached to different aglycons and also some longer oligosaccharides revealed that Brainiac preferentially utilized βMan containing structures, while αMan terminated structures were poor acceptors. Interestingly, while D-galactose and βGal-aglycans were not found to be acceptors, βGal terminating disaccharides including lactose and benzyl-lactose (Galβ1-4Glc) as well as Galβ1-4Man structures. In the latter case the acceptor sugar was not determined. Table I summarizes the results:

TABLE I
Substrate specificities of Brainiac β1-3-N-acetylglucosaminyltransferase

| | Brainiac ^{a} | |
|---|---|---|
| | nmol/h/mg | |
| Substrate | 2 mM | 20 mM |
| D-Man | 0.28 | 1.70 |
| Manα1 -Me | 0.36 | 0.61 |
| Manβ1-Me | 1.46 | 2.74 |
| Manα1-MeUmb | 0.11 | 0.17 |
| Manβ1-MeUmb | 5.25 | 6.18 |
| Manβ1-2Manα1-Me | 0.15 | 0.16 |
| Manα1-3Manα1-Me | 0.00 | 0.00 |
| Manα1-4Manα1-Me | 0.00 | 0.00 |
| Manα1-6Manα1-Me | 0.02 | 0.05 |
| Manα1-6(Manα1-3)Manα1-Bzl | 0.01 | 0.08 |
| Manα1-3(Manα1-6)Manα1-6(Manα1-3)Man | 0.09 | 0.45 |
| Manβ1-4GlcNAc | ND ^{b} | ND |
| D-Gal | 0.00 | 0.00 |
| Galβ1-MeUmb | 0.19 | 0.00 |
| Galβ1-oNph | 0.21 | 0.02 |
| Galβ1-4Glc | 0.42 | 3.08 |
| Galβ1-4Glcβ1-Bzl | 0.48 | 1.69 |
| Galβ1-4GlcNAc | 0.03 | 0.12 |
| Galβ1-4GlcNAcβ1-Bzl | 0.04 | 0.02 |
| Galβ1-4Man | 1.48 | 8.39 |
| Galβ1-4ManNAc | 0.10 | 0.42 |
| Galβ1-4Gal | 0.00 | 0.00 |
| Galβ1-4Fru | 0.05 | 0.18 |
| lacto-N-tetraose | 0.00 | 0.00 |
| lacto-N-neo-tetraose | 0.03 | ND |
| D-Fuc | 0.01 | 0.01 |
| L-Fuc | 0.00 | 0.05 |
| L-Fucosylamine | 0.28 | 0.60 |
| L-Fucβ1-pNph | 0.01 | 0.02 |
| L-Fucα1-pNph | 0.02 | 0.03 |
| L-Fucα1-MeUmb | 0.00 | 0.00 |
| D-Fucα1-MeUmb | 0.00 | 0.00 |

^{a} Enzyme sources were microsomal preparations of pVL-Brainiac infected High Five ™ cells (see “Experimental Procedures”). Background values obtained with microsomes of cells infected with an irrelevant construct were subtracted.
^{b} ND not determined.

### Example 8

Brainiac encodes a β1,3N-acetylglucosaminyltransferase functioning in glycolipid biosynthesis.

Glycosphingolipids of insect cells have been reported to have the internal core structure GlcNAcβ1-3Manβ1-4Glcβ1-Cer, with a βmannose residue (Seppo A, Moreland M, Schweingruber H, Tiemeyer M. Zwitterionic and acidic glycosphingolipids of the Drosophila melanogaster embryo. Eur J Biochem. 2000;267:3549-58.). βMannose residues are not found in glycosphingolipids of higher eukaryotic cells where all elongated glycosphingolipid species are built on Galβ1-4Glcβ1-Cer. Thus, the glycolipid structure Manβ1-4Glcβ1-Cer represented a possible substrate for Brainiac based on the acceptor sugar specificity identified in Example 7.

The glycolipid Manβ1-4Glcβ1-Cer (Mac-Cer/CDH) was purified from High Five cells and demonstrated to serve as an efficient substrate for Brainiac (FIG. 8). In accordance with the saccharide specificity of Brainiac the glycolipid Galβ1-4Glcβ1-Cer (Lac-Cer/CDH) also served as a substrate.

In higher eukaryotic cells the most essential diverging step in glycolipid biosynthesis is the addition of the third sugar residue to form lactoseries (GlcNAcβ1-3Galβ1-4Glcβ1-Cer), globoseries (Galα1-4/3Galβ1-4Glcβ1-Cer), and ganglioseries (GalNAcβ1-4Galβ1-4Glcβ1-Cer) glycosphingolipid structures. Brainiac is shown to serve one of these functions to direct lactoseries synthesis with Lac-Cer and in insect cells Mac-Cer. Reports of structure analysis of glycolipids in insects have not yet described other species than those carried on GlcNAcβ1-3Manβ1-4Glcβ1-Cer, however, we have identified Galβ1-4Manβ1-4Glcβ1-Cer as the major CTH migrating glycolipid in High Five insect cells (data not shown). It is therefore conceivable that Brainiac controls one of multiple divergent pathways of glycolipid biosynthesis in insect cells as well.

It is interesting to note that Brainiac also functions with Lac-Cer, a glycolipid which has never been described from insects. Brainiac is the ancestral gene of a very large homologous β3glycosyltransferase gene family in mammalian cells, and the members of this family with closest sequence similarity to Brainiac is represented by a family of β3GlcNAc-transferases with similar specificity for Lac-Cer (Shiraishi et al., 2001, J. Biol. Chem., 276: 3498-3507.). One of these designated human β3GnT2 was analysed in detail and shown to use Lac-Cer but not Mac-Cer (not shown).

Brainiac's role in glycolipid biosynthesis is likely to play a role in receptor signaling. Glycolipids in mammalian cells have long been known to modulate cell surface receptors and cell signaling (see Hakomori and Igarashi (1995), J. Biochem. (Tokyo); 118:1091-103.; Meuillet et al., 2000, Exp. Cell Res.;256: 74-82.). Although the specific role of Brainiac in this regard has not been demonstrated in insect cells, it is clear that its role in glycolipid biosynthesis could control glycolipid expression and the functions of particular glycolipid species in receptor modulation and cell signaling. Such particular glycolipid species have not been defined in insect cells, but an example of these are certain gangliosides in mammalian cells. Since the biosynthesis of longer gangliosides in mammalian cells require the structure GalNAcβ1-4Galβ1-4Glcβ1-Cer, it is clear that a βGalNAc-transferase has to compete with β3GlcNAc-transferases and others enzymes in order to synthesize the ganglisoseries pathway. Thus, the enzymes controlling the third step in the biosynthesis of glycolipids may play an important role in directing signals through glycolipid induced modulation of receptor functions.

Conclusion

Taken together our results provide evidence that Fringe is a glycosyltransferase that acts in the Golgi to modify Notch, resulting in altered ligand binding specificity. Fringe is likely to determine the type of O-linked Fucose extension on the EGF-repeats of Notch, and possibly on other EGF-repeat-containing proteins. Fringe shows some sequence similarity to Brainiac. Brainiac is shown to catalyse a similar reaction, but in addition, to catalyse reactions that are specific for synthesis of a specific class of glycolipid.

Large families of proteins related to Brainiac and Fringe have been identified in vertebrates. Proteins that are distantly related to Brainiac (Röttger et al., 1998) have been characterised as β3Gal or β3GlcNac glycosyltransferases with different acceptor substrate specificities (see Amado et al., 1999). Those characterised to date function in N-glycosylation, mucin type O-glycosylation and glycolipid biosynthesis, but not O-linked glycoprotein modification. Evidence for an enzyme activity capable of elongating O-linked Fucose by addition of Glucose has also been reported recently. It therefore seems possible that members of the Brainiac family and other glycosytransferases might also be able influence receptor function.

Drosophila Brainiac has been implicated as a modulator the activities of Notch and of the EGF receptor (Goode et al., 1997). Fringe-mediated modification changes the properties of Notch-Delta binding and has an important role in conferring signalling specificity in vivo. We suggest that this and other oligosaccharide side-chain modifications may open up a new range of possibilities for regulation of ligand-receptor interactions in a cell-type and protein specific manner.

References

Amado, M. et al. A family of human beta3-galactosyltransferases. Characterisation of four members of a UDP-galactose:beta-N-acetyl-glucosamine/beta-N acetyl-galactosamine beta-1,3-galactosyltransferase family. J Biol Chem 273, 12770-8 (1998).

Amado, M., Almeida, R., Schwientek, T. & Clausen, H. Identification and characterisation of large galactosyltransferase gene families: galactosyltransferases for all functions. Biochim. Biophys. Acta 1473, 35-53 (1999).

Artavanis-Tsakonas, S., Rand, M. D. & Lake, R. J. Notch signalling: cell fate control and signal integration in development. Science 284, 770-6 (1999).

Bennett, E. P., Hassan, H. & Clausen, H. cDNA cloning and expression of a novel human UDP-N-acetyl-alpha-D-galactosamine: Polypeptide N-acetylgalactosaminyltransferase, GalNAc-t3. J Biol Chem 271, 17006-17012 (1996).

Bergemann, A. D., Cheng, H. J., Brambilla, R., Klein, R. & Flanagan, J. G. ELF-2, a new member of the Eph ligand family, is segmentally expressed in mouse embryos in the region of the hindbrain and newly forming somites. Mol Cell Biol 15, 4921-9 (1995).

Blaumueller, C. M., Qi, H., Zagouras, P. & Artavanis-Tsakonas, S. Intracellular cleavage of Notch leads to a heterodimeric receptor on the plasma membrane. Cell 90, 281-291 (1997).

Breton, C. & Imberty, A. Structure/function studies of glycosyltransferases. Curr Opin Struct Biol 9, 563-71 (1999).

Bruckner, K. et al. EphrinB ligands recruit GRIP family PDZ adaptor proteins into raft membrane microdomains. Neuron 22, 511-24 (1999).

Bruckner, K., Pasquale, E. B. & Klein, R. Tyrosine phosphorylation of transmembrane ligands for Eph receptors. Science 275, 1640-3 (1997).

Cheng, H. J. & Flanagan, J. G. Identification and cloning of ELF-1, a developmentally expressed ligand for the Mek4 and Sek receptor tyrosine kinases. Cell 79, 157-68 (1994).

Fehon, R. G. et al. Molecular interactions between the protein products of the neurogenic loci Notch and Delta, two EGF-homolgous genes in Drosophila. Cell 61, 523-534 (1990).

Fleming et al., Trends Cell Biol. 7, 437-441 (1997).

Gastinel, L. N., Cambillau, C. & Bourne, Y. Crystal structures of the bovine beta4galactosyltransferase catalytic domain and its complex with uridine diphosphogalactose. Embo J 18, 3546-57 (1999).

Goode et al., Dev. Biol. 178, 35-50 (1996).

Goode, S. & Perrimon, N. Brainiac and fringe are similar pioneer proteins that impart specificity to notch signalling during Drosophila development. Cold Spring Harb Symp Quaint Biol 62, 177-84 (1997).

Hagen, F. K. Hazes, B., Raffo, R., deSa, D. & Tabak, L. A. Structure-function analysis of the UDP-N-acetyl-D-galactosamine:polypeptide N-acetylgalactosaminyltransferase. Essential residues lie in a predicted active site cleft resembling a lactose repressor fold. J Biol Chlem 274, 6797-803 (1999).

Harris, R. J., Van Halbeek, H., Glushka, J., Basa, L. J., Ling, V. T., Smith, K. J., and Spellman, M. W. (1993) Biochemistry 32, 6539-6547.

Harris, R. J. & Spellman, M. W. O-linked fucose and other post-translational modifications unique to EGF modules. Glycobiology 3, 219-224 (1993).

Jeanmougin, F., Thompson, J. D., Gouy, M., Higgins, D. G. & Gibson, T. J. (1998). Multiple sequence alignment with Clustal X. Trends Biochem Sci 23, 403-5.

Kim, J., Irvine, K. D. & Carroll, S. B. Cell recognition, signal induction and symmetrical gene activation at the dorsal/ventral boundary of the developing Drosophila wing. Cell 82, 795-802 (1995).

Logeat, F. et al. The Notchl receptor is cleaved constitutively by a furin-like convertase. Proc Natl Acad Sci USA 95, 8108-12 (1998).

Minamida, S., Aoki, K., Natsuka, S., Omichi, K., Fukase, K., Kusumoto, S., and Hase, S. (1996) J. Biochem. (Tokyo) 120, 1002-1006.

Moloney, D. J., Lin, A. I., and Haltiwanger, R. S. (1997) J. Biol. Client. 272, 19046-19050.

Moloney, D. J. & Haltiwanger, R. S. The O-l fucose glycosylation pathway: identification and characterisation of a uridine diphosphoglucose: fucose-botal, 3-glucosyltransferase activity from Chinese hamster ovary cells. Glycobiology 9, 679-687 (1999).

Moloney et al., J. Biol. Chem. 275(13), 9604-9611.

Nilsson, T. & Warren, G. Retention and retrieval in the endoplasmic reticulum and the Golgi apparatus. Curr. Opin. Cell Biol. 6, 517-521 (1994).

Nishimura, H., Kawabata, S., Kisiel, W., Hase, S., Ikenaka, T., Takao, T., Shimonishi, Y., and Iwanaga, S. (1989) J. Biol. Chem. 264,20320-20325.

Nishimura, H., Takao, T., Hase, S., Shimonishi, Y., and Iwanaga, S. (1992) J. Biol. Chem. 267, 17520-17525.

Omichi, K., Aoki, K., Minamida, S., and Hase, S. (1997) Eur. J. Biochem. 245, 143-146.

Panin, V. M., Papayannopoulos, V., Wilson, R. & Irvine, K. D. Fringe modulates Notch-ligand interactions. Nature 387, 908-913 (1997).

Röttger, S. et al. Localisation of three human polypeptide GalNAc-transferases in HeLa cells suggests initiation of O-linked glycosylation throughout the Golgi apparatus. J Cell Sci 111, 45-60 (1998).

Schaffer, A. A., Wolf, Y. I., Ponting, C. P., Koonin, E. V., Aravind, L. & Altschul, S. F. (1999). IMPALA: matching a protein sequence against a collection of PSI-BLAST-constructed position-specific score matrices. Bioinformatics 15, 1000-11.

Shao, L., Moloney, D. J., and Haltiwanger, R. S. (1998) Glycobiology 8, 1110.

Strigini, M. & Cohen, S. M. A Hedgehog activity gradient contributes to AP axial patterning of the Drosophila wing. Development 124, 4697-4705 (1997).

Wang, Y., and Spellman, M. W. (1998) J. Biol. Chem. 273, 8112-8118.

Wu, J. Y., Wen, L., Zhang, W. J. & Rao, Y. The secreted product of Xenopus gene lunatic Fringe, a vertebrate signalling molecule. Science 273, 355-358 (1996).

Yuan, Y. P., Schultz, J., Mlodzik, M. & Bork, P. Secreted fringe-like signalling molecules may be glycosyltransferases. Cell 88, 9-11 (1997).

1. Use of a Brainiac protein, or a fragment, or functional equivalent of a Brainiac protein, as a glycosyltransferase, wherein said Brainiac protein, fragment thereof, or functional equivalent thereof acts to elongate a mannose residue linked to a protein via an O-linked glucose residue or via a ceramide in a glycolipid.

2. Use according to claim 1, wherein said Brainiac protein, or fragment, or functional equivalent of a Brainiac protein acts us a glycosyltransferase on an EGF-module containing protein.

3. Use according to claim 2, wherein said an EGF-module containing protein is a Notch protein or a protein in the Notch signalling pathway.

4. Use according to claim 3, wherein said Notch protein or protein in the Notch signalling. pathway is, or is a functional equivalent of Notch 2 (gi|1082649 |pir||A56695 [1082649]); Notch 3 ([Homo sapiens] gi|2668592| gb|AAB91371.1|[2668592]); Notch4 ([Homo sapiens] gi|2072309| gb|AAC32288.1| [2072309]); or the Notch protein homologue TAN-1 precursor (human gi|107215|pir||A40043[107215]).

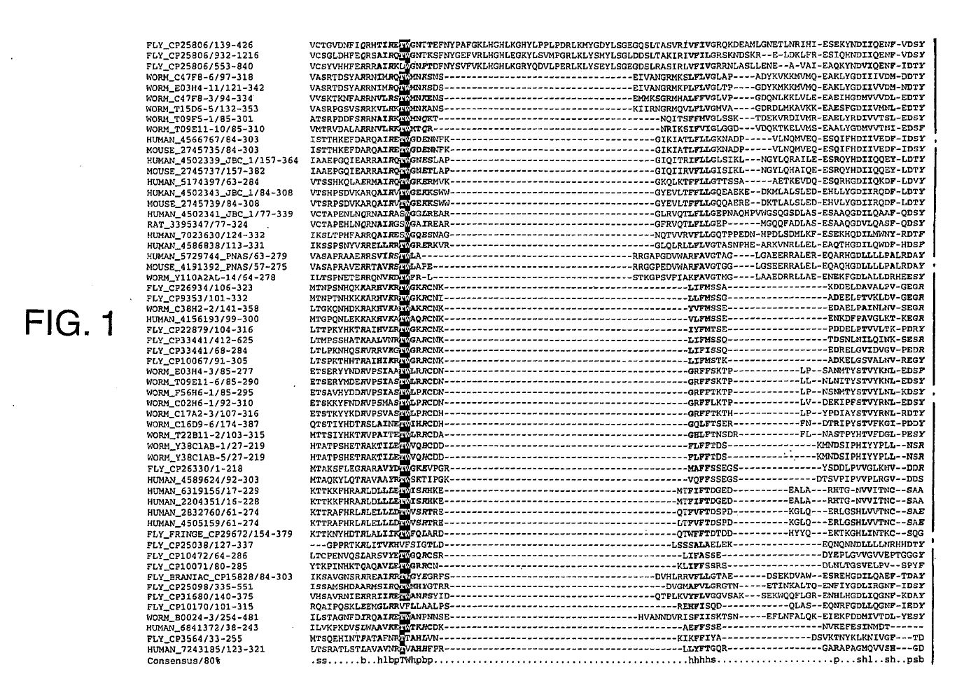

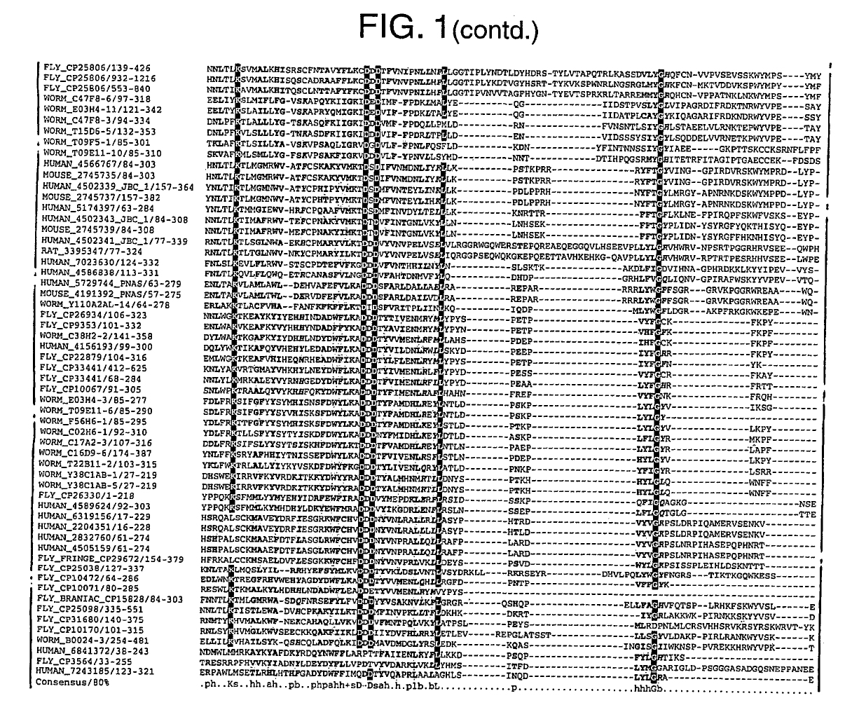

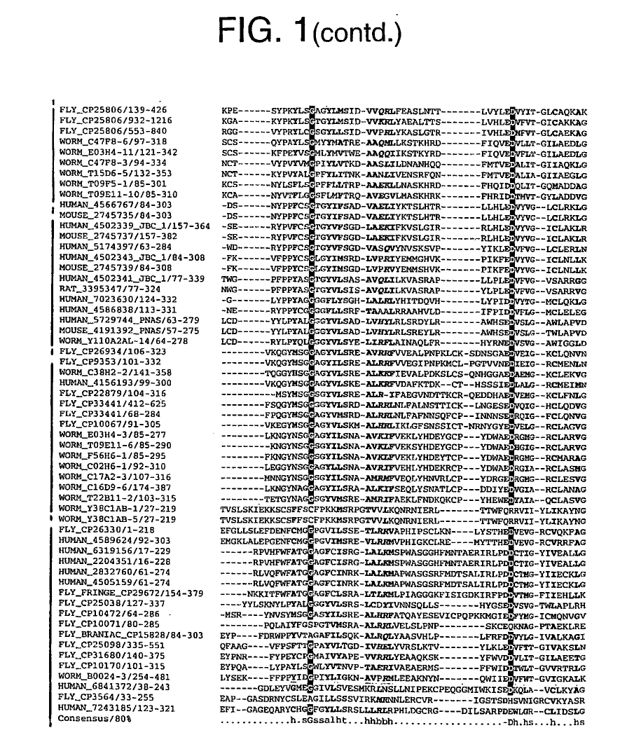

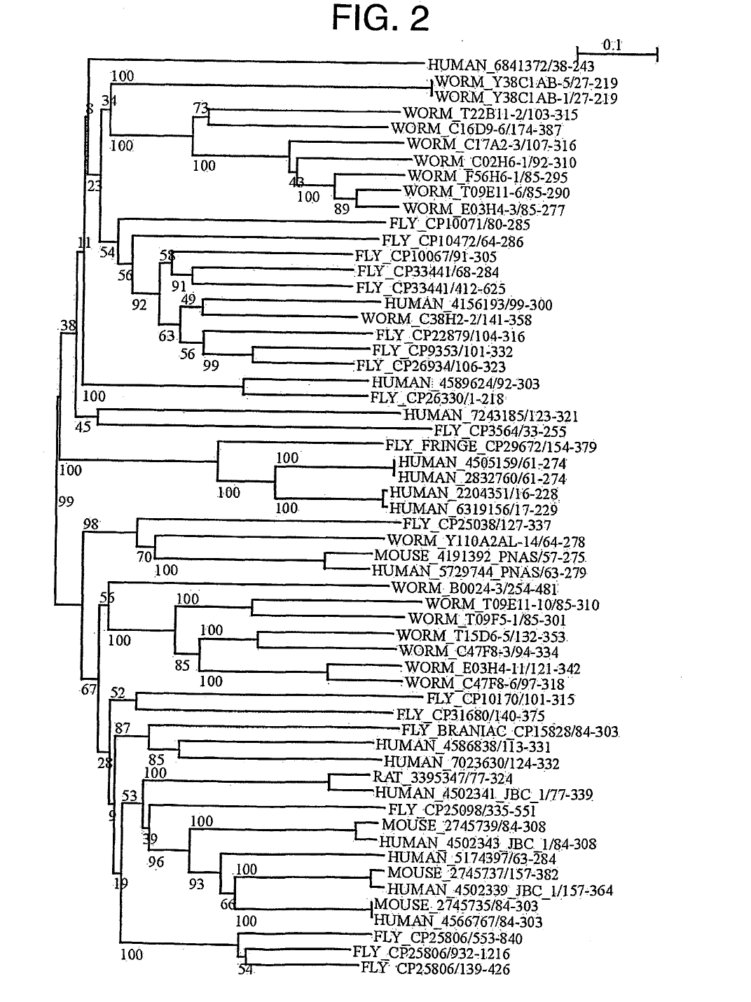

5. Use according to any one of claims 1-4, wherein said Brainiac protein is any one of the Drosophila proteins identified by the accession codes: gb|AAF48225.1| (AE003491) CG4351 gene product; gb|AAF52606.1| (AE003620) CG8668 gene product; gb|AAF47918.1| (AE003481) CG11357 gene product; gb|AAF58600.1| (AE003824) CG8976 gene product; gb|AAF59065.1| (AE003836) CG8734 gene product; gb|AAF59121.1| (AE003838) CG8708 gene product; gb|AAF47429.1| (AE003469) CG13904 gene product; gb|AAF48225.1| (AE003491) CG4351 gene product; a functional homologue identified in either of FIGS. 1 and/or 2, or a functional homologue that shares similarity according to the criteria used to build the phylogenetic tree shown in FIG. 2.

6. Use of a ligand of a Brainiac protein as a glycosyltransferase inhibitor, wherein said ligand is effective to prevent elongation of a mannose residue linked to a protein via an O-linked glucose residue or via a ceramide in a glycolipid.

7. Use according to claim 6, wherein said mannose residue is via an O-linked glucose residue to an EGF-module containing protein.

8. Use according to claim 7, wherein said prevention of glycosylation has an effect on the binding of an effector protein to an EGF-module containing protein

9. Use according to claim 7 or claim 8, wherein said EGF-module containing protein is a Notch protein or a protein in the Notch signalling pathway.

10. Use according to claim 9, wherein said effector protein is, or is a mammalian homologue of Delta, Delta-like, Jagged, Serrate or any other Notch ligand.

11. Use of a Brainiac protein, or a fragment or functional equivalent of a Brainiac protein, or of a ligand of a Brainiac protein, in the manufacture of a medicament for the treatment of a disease caused by a protein containing one or more EGF-like modules, in which said disease is characterised by a deficiency in glycosylation of mannose residues linked to the protein via an O-linked glucose residue or in glycosylation of mannose residues linked to via a ceramide in a glycolipid.

12. Use according to claim 11, wherein said protein containing one or more EGF-like modules is a Notch protein, or a protein in the Notch signalling pathway.

13. Use according to clam 11 or claim 12, wherein said disease is T cell leukaemia, breast cancer, stroke, dementia, cerebral autosomal dominant arteriopathy with subcortical infarcts, leukoencephalopathy or Alagille syndrome.

14. A method of treating a disease caused by an EGF-like module containing protein, in which there is a deficiency in glycosylation of mannose residues linked to the protein via an O-linked glucose residue or in glycosylation of mannose residues linked to via a ceramide in a glycolipid, said method comprising administering to a patient a Brainiac protein, or a fragment, or functional equivalent of a Brainiac protein, or a ligand of a Brainiac protein.

15. A Brainiac protein, or a fragment or functional equivalent of a Brainiac protein, for use as a glycosyltransferase, wherein said Brainiac protein, fragment thereof, or functional equivalent thereof acts to elongate a mannose residue linked to a protein via an O-linked glucose residue or via a ceramide in a glycolipid.

16. A method of transferring a N-acetylglucosamine moiety onto a mannose substrate, whether free or attached to a lipid, carbohydrate or protein, comprising the step of incubating a Brainiac protein, or a fragment, or functional equivalent of a Brainiac protein with said substrate.

17. A method of screening for a ligand capable of modulating the activity of a Brainiac protein, said method comprising the steps of
a) contacting a candidate ligand with said Brainiac protein or a fragment thereof; and
b) testing the effect of the ligand on the glycosyltransferase activity of said Brainiac protein, wherein said Brainiac protein or fragment thereof acts to elongate a mannose residue linked to a protein via an O-linked glucose residue or via a ceramide in a glycolipid.

18. A method of screening for a ligand of a substrate for a Brainiac protein, said method comprising the steps of:
a) contacting a candidate ligand with said substrate in the presence of a Brainiac protein; and
b) testing the effect of the ligand on the extent of elongation of mannose residues linked to a protein substrate via an O-linked glucose residue or via a ceramide in a glycolipid substrate.

19. A method according to claim 18, wherein said substrate is a polypeptide comprising at least one EGF module.

20. A method for the identification of a gene that is implicated in a disease or physiological condition in which Brainiac function plays a role, said method comprising the steps of:
a) comparing:
i) the transcriptome or proteome of a first cell type; with
ii) the transcriptome or proteome of a second cell type in which the expression or activity of the Brainiac protein is altered in comparison to the first cell type; and
b) identifying as the gene implicated in the disease or condition, a gene encoding a protein whose level of glycosylation differs between the two cell types, wherein said level of glycosylation is the extent of elongation of mannose residues linked to a protein via an O-linked glucose residue or via a ceramide in a glycolipid.
